(19) United States
(12) Patent Application Publication
(10) Pub. No.: US 2011/0028959 A1
(43) Pub. Date: Feb. 3, 2011

(54) Surgical Apparatus and Method for Performing Minimally Invasive Surgery

(76) Inventor: Paul Chasan, San Diego, CA (US)

Correspondence Address: FISH & RICHARDSON P.C. (SD), P.O. BOX 1022, MINNEAPOLIS, MN, 55440-1022, US

(21) Appl. No.: 12/512,750

(22) Filed: Jul. 30, 2009

(51) Int. Cl. A61B 18/20; A61B 18/18; A61B 18/14; A61B 17/32; A61B 18/04

(52) U.S. Cl. 606/13; 606/33; 606/49; 606/170; 606/27

(57) ABSTRACT

An apparatus for performing minimally invasive surgery, for example, percutaneous minimally invasive capsulotomy surgery. The apparatus includes a shaft, a blunt tip, a flange, an energy conductor and an active surgical element, which may be a cutting or heating element. The shaft has a first end and a second end and is generally stiff with a degree of flexure; the shaft may be curved along at least a substantial part of its length to facilitate navigating the blunt tip within a patient's body. The blunt tip is at the second end of the shaft and may be larger in diameter than the shaft and with a recessed area along a surface of the blunt tip. The flange is located at the second end of the shaft and is configured to receive a device that delivers energy, which may be in the form of heat, laser light, radio-frequency, or electricity. The energy conductor is disposed along the length of the shaft from the second end of the shaft to the recess, the energy conductor configured to conduct the energy. The active surgical element, whether it be a cutting or heating element, is located within the recessed area of the blunt tip and coupled to the energy conductor and is configured to make incisions in response to receiving the energy. Also disclosed are methods for performing various surgical procedures using the surgical apparatus, including percutaneous minimally invasive capsulotomy surgery.

### TECHNICAL FIELD

This invention relates to the medical field, and more particularly to an apparatus for performing minimally invasive surgery.

### BACKGROUND

Traditionally, surgeons performed open surgery on patients requiring cutting of the skin and tissue to provide the surgeon with direct access to the structures or organs involved in the surgical procedure. The target of the surgery, such as a patient's joint or an organ, can then be seen and touched.

Over the years, many surgical procedures have become less invasive, using smaller incisions and less dissection and trauma to tissues, allowing the patient to recover in less time and with less pain and scarring. These surgical procedures are frequently referred to as minimally invasive surgical procedures. Minimally invasive surgical procedures often use special surgical instruments that may be used to manipulate, cut, suture, and/or cauterize tissues. The surgical instruments generally include a small diameter device that can be inserted into a patient through a small incision that requires only a few sutures (or perhaps just one) to close. Examples of minimally invasive surgical techniques include endoscopy, arthroscopy, and laparoscopy.

Minimally invasive surgical procedures have been employed in orthopedic and plastic surgeries. For example, arthroscopy is minimally invasive procedure used by orthopedic surgeons to evaluate or treat many orthopedic conditions including torn cartilage, torn surface cartilage, and ACL reconstruction. Plastic surgeons also have begun to experiment with and employ minimally invasive surgical techniques. However, there are still many orthopedic and plastic surgical procedures that cannot currently be performed in a minimally invasive fashion. Instead, surgeons must use traditional open surgical techniques, resulting in longer recovery time and more scarring.

Therefore, a need exists for new surgical devices that would allow more surgical procedures to be performed in a minimally invasive fashion. The present invention provides such a device.

### SUMMARY

The details of one or more embodiments of the invention are set forth in the accompanying drawings and the description below. Other features, objects, and advantages of the invention will be apparent from the description and drawings, and from the claims.

An embodiment of the present invention is an apparatus for performing minimally invasive surgery that includes a shaft, a blunt tip, a flange, an energy conductor, and an active surgical element, which may be a cutting or heating element. The shaft has a first end and a second end and is generally stiff with a degree of flexure. The blunt tip is located at the second end of the shaft, the rounded blunt tip being larger in diameter than the shaft and having an active surface and a non-active surface and a recessed area along the active surface. The flange is located at the second end of the shaft and is configured to receive a device that delivers an energy. The energy conductor is disposed along the length of the shaft from the second end of the shaft to the recess and is configured to conduct the energy. The active surgical element, whether it be a cutting or heating element, is located within the recessed area of the rounded blunt tip and coupled to the energy conductor, the active surgical element configured to surgically modify tissue in response to receiving the energy from the energy conductor. If the active surgical element is a cutting element, the tissue will be modified by cutting; if a heating element, the modification may be shrinking of the tissue.

In another embodiment, the present invention is an apparatus for performing minimally invasive surgery that includes a shaft, a blunt tip means, a flange means, a conductor means, and a cutting means. The shaft has a first end and a second end, the shaft being generally stiff with a degree of flexure. The blunt tip means is located at the second end of the shaft and having a cutting surface and a non-cutting surface and is for inserting into a patient's body through a small incision. The flange means is located at the second end of the shaft and is for receiving a device that delivers an energy. The conductor means is disposed along the length of the shaft from the second end of the shaft to the blunt tip means and is for conducting the energy to the blunt tip means. The cutting means is located on the cutting surface of the blunt tip means and responsive to the energy from the conductor means and is for making incisions in the patient from the cutting surface of the blunt tip means and away from the non-cutting surface of the blunt tip means.

In another embodiment, the present invention is a minimally invasive method for performing capsulotomy surgery. The method includes providing a surgical device having a shaft, a blunt tip, and a flange. The shaft has a first end and a second end, the shaft being generally stiff with a small amount of flexure. The blunt tip is located at the second end of the shaft and is larger in diameter than the shaft and has a cutting side and a non-cutting side, the blunt tip having a recess located on the cutting side. A surgical cutting element is located within the recess of the blunt tip and is directed away from the non-cutting side. An energy conductor is disposed along the length of the shaft from the second end of the shaft to the surgical cutting element and is configured to deliver an energy for use by the surgical cutting element. The flange is located at the second end of the shaft and is configured to receive a device that delivers the energy to the energy conductor. The method further comprises inserting the blunt tip of the surgical device into a small incision on or near a breast of a patient and into a cavity surrounding a breast implant within the breast; passing the blunt tip between the implant and surrounding scar tissue (capsular tissue) and directing the cutting side and the surgical cutting element toward the scar tissue and away from the implant; and cutting the scar tissue with the surgical cutting element.

In yet another embodiment, the present invention is a minimally invasive method for performing spinal surgery that includes providing a surgical device having a shaft, a blunt tip, and a flange. The shaft has a first end and a second end, the shaft being generally stiff with a small amount of flexure. The blunt tip is located at the second end of the shaft and is larger in diameter than the shaft and has a cutting side and a non-cutting side, the blunt tip having a recess located on the cutting side. A surgical cutting element is located within the recess of the blunt tip and is directed away from the non-cutting side. An energy conductor is disposed along the length of the shaft from the second end of the shaft to the surgical cutting element and is configured to deliver an energy for use by the surgical cutting element. The flange is located at the second end of the shaft and is configured to receive a device that delivers the energy to the energy conductor. The method further includes inserting the blunt tip of the surgical device into a small incision on or near a patient's spine; directing the blunt tip toward a scar tissue disposed at or near a nerve or nerve root; directing the cutting surface of the blunt tip toward the scar tissue and the non-cutting surface of the blunt tip toward the nerve or nerve root; and cutting the scar tissue with the cutting element of the surgical device.

In another embodiment, the present invention is a minimally invasive method for performing joint surgery. The method includes providing a surgical device having a shaft, a blunt tip, and a flange. The shaft has a first end and a second end, the shaft being generally stiff with a small amount of flexure. The blunt tip is located at the second end of the shaft and is larger in diameter than the shaft and has a heating side and a non-heating side, the blunt tip having a recess located on the heating side. A surgical heating element is located within the recess of the blunt tip and is directed away from the non-heating side. An energy conductor is disposed along the length of the shaft from the second end of the shaft to the surgical heating element and is configured to deliver an energy for use by the surgical heating element. The flange is located at the second end of the shaft and is configured to receive a device that delivers the energy to the energy conductor. The method further includes inserting the blunt tip of the surgical device into a small incision on or near a patient's joint; directing the blunt tip toward a lining or capsule of the joint; directing the heating surface of the blunt tip toward the lining or capsule and the and the non-heating surface of the blunt tip away from the lining or capsule; and heating the lining or capsule with the surgical heating element of the surgical device.

### DESCRIPTION OF DRAWINGS

FIG. 1 is a perspective view of an embodiment of the surgical device of the present invention.

FIG. 2 is a top view of an embodiment of the surgical device of the present invention.

FIG. 3 is a side view of an embodiment of the surgical device of the present invention.

FIG. 4 is a cut-away side view of an embodiment of the surgical device of the present invention, taken along line A-A in FIG. 2.

FIG. 5 is cross-sectional view of an embodiment of the blunt tip of the surgical device of the present invention viewed from the front of the blunt tip, along line B-B shown in the top view of FIG. 2.

FIG. 6 is a front view of an embodiment of the surgical device of the present invention.

FIG. 7 is a top view of an embodiment of the blunt tip of the surgical device of the present invention taken along line C shown in the top view of FIG. 2.

FIG. 8 is a front view of an embodiment of the blunt tip of the surgical device of the present invention taken along line D in the top view of FIG. 6.

FIG. 9A is a side view of a patient showing how an embodiment of the surgical device can be used to perform percutaneous minimally invasive capsulotomy surgery.

FIG. 9B is a front view of a patient showing how an embodiment of the surgical device can be used to perform percutaneous minimally invasive capsulotomy surgery.

Like reference symbols in the various drawings indicate like elements.

### DETAILED DESCRIPTION

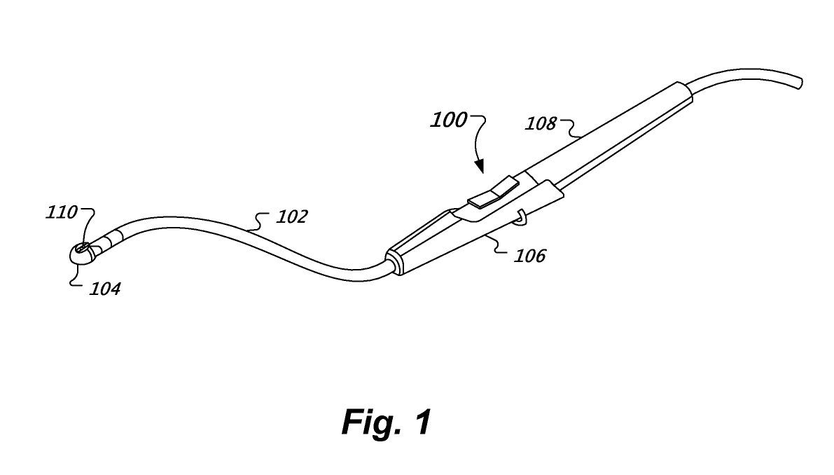

FIG. 1 shows an embodiment of the surgical device 100 of the present invention. The device 100 includes a shaft 102, a blunt tip 104, and a flange 106. The flange is configured to receive an energy delivery mechanism 108 that provides a form of energy, such as electricity, radio-frequency, heat, or light. The shaft 102 is generally stiff with a degree of flexure. A shaft that is generally stiff allows the surgeon to easily move the blunt tip 104 around in a patient's body, while the degree of flexure allows the shaft 104 to bend around and avoid structures within the patient's body (including curving around a breast implant) when the blunt tip 104 comes into contact with such structures. The shaft 102 may have only a small degree of flexure, so that the shaft 102 remains generally rigid and can be navigated easily within the patient's body. Those skilled in the art will appreciate and understand that the shaft 102 may be designed with different degrees of stiffness and flexure depending on the surgical procedure for which the surgical device 100 intended. For example, the flexure may be similar to that of a bamboo shoot. As shown in the embodiment of FIG. 1, the shaft 102 is curved along its length, and in particular in this embodiment, the curvature occurs at two locations along the shaft 102. The curvature of the shaft 102 further facilitates ease of movement of the blunt tip 104 within the patient's body. Those skilled in the art will appreciate and understand that the shaft 102 may be designed with different curvatures depending on the surgical procedure for which the surgical device 100 intended.

The blunt tip 104, as embodied in FIG. 1, may be rounded and slightly larger in diameter than the shaft 104. Those skilled in the art will appreciate and understand, however, that the blunt tip 104 need not be rounded and/or may be set at different angles to achieve the goal. The blunt tip 104 includes a surgical cutting element 110 that may be used to make incisions or cuts in tissue, organs, or other structures within the patient's body. In an embodiment of the invention, the surgical cutting element 110 may be a radio-frequency cautery element that cauterizes and thereby cuts tissue, organs, or other structures. If a cautery element is employed, the energy delivery mechanism may be a standard surgical cautery device that can be plugged into a standard socket in the flange 106. A conductor (shown in FIG. 4, reference numeral 112) may be disposed along the length of the shaft 102 from the flange 106 to a position at or proximate the blunt tip 104 to provide energy for the standard cautery device to the cautery element on the blunt tip 104, and the conductor 112 may be coupled to the surgical cutting element 110 to deliver energy the element 110. In this way, the surgical device 100 can be easily used by any surgeon with access to a standard cautery device. Alternatively, the surgical cutting element 110 may be a laser element that makes incisions using laser light. If a laser element is used, the energy delivery mechanism 108 may be a standard surgical laser device that delivers laser light energy along the conductor to the laser element on the blunt tip 104. The flange 106, in this embodiment, would be configured to receive the standard surgical laser device, again making it simple for any surgeon with access to a standard surgical laser device to use the surgical device 100 of the present invention. With the energy delivery mechanism 108 inserted into the flange 106, the two elements create a convenient and comfortable handle for the surgeon to get a firm grip on the surgical device 100.

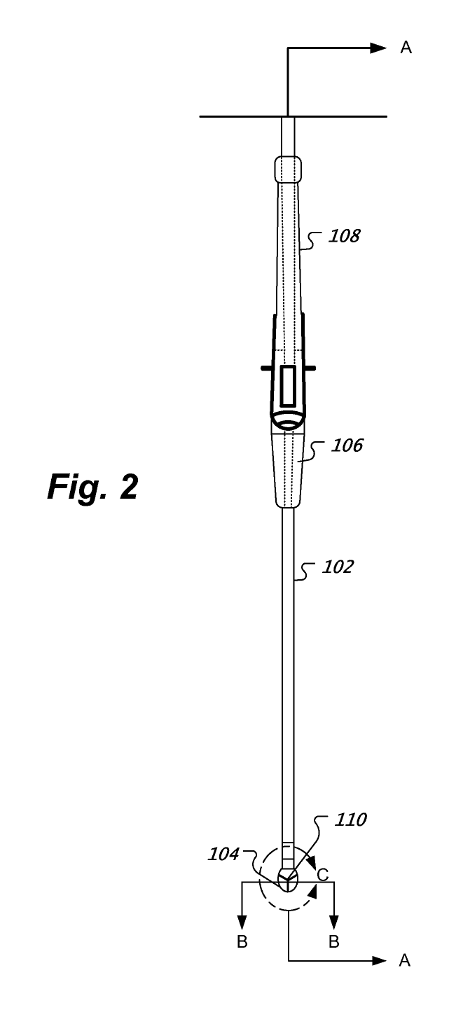

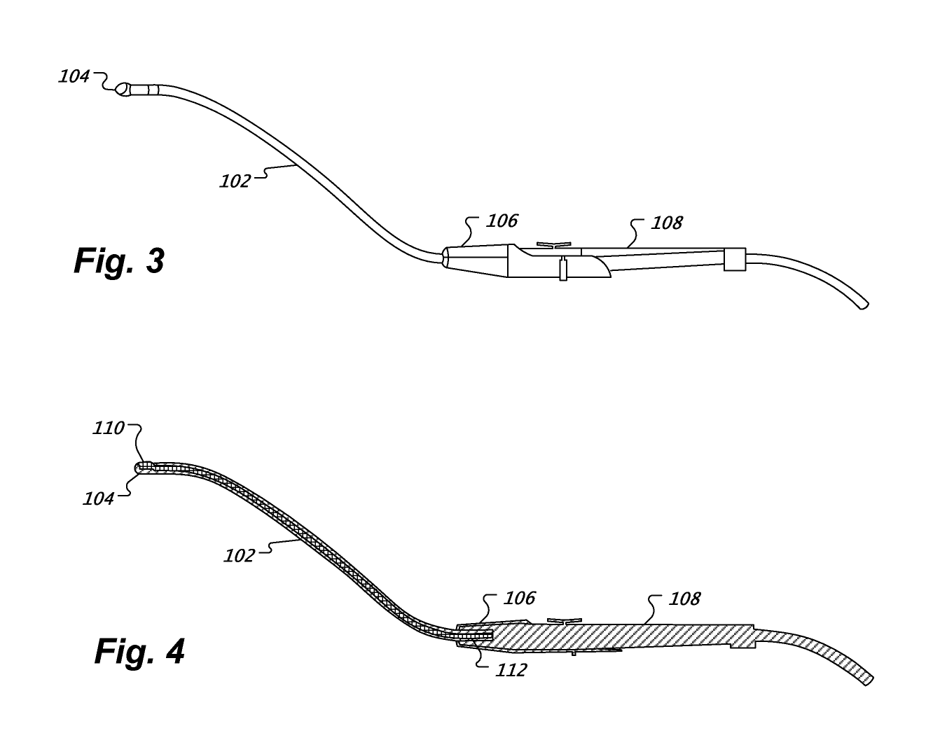

FIG. 2 is top view of the surgical device 100, while FIG. 3 is a side view of the surgical device 100. Both of these figures show the shaft 102, blunt tip 104, flange 106, and energy delivery mechanism 108. The figures also show how the flange 106 is designed to receive the energy delivery mechanism 108. In addition, the figures illustrate the exemplary curvature of the shaft 102. As noted above, the shaft 102 may be curved in different ways, or may not be curved at all. The top view in FIG. 2 illustrates that the surgical cutting element 110 may be located on a side of the blunt tip 104.

FIG. 4 is a cut-away side view of the embodiment of the surgical device 100 shown in FIG. 1. FIG. 4 shows a cut-away view of the shaft 102, the blunt tip 104, the flange 106, the energy delivery mechanism 108 inserted into the flange 106, and the surgical cutting element 110. FIG. 4 also shows a conductor 112 that may be disposed along the length of the shaft 102 from the flange 106 to the blunt tip 104. The conductor may be used to conduct energy, such as electricity, heat, or light, from the energy delivery mechanism 108 to the surgical cutting element 110.

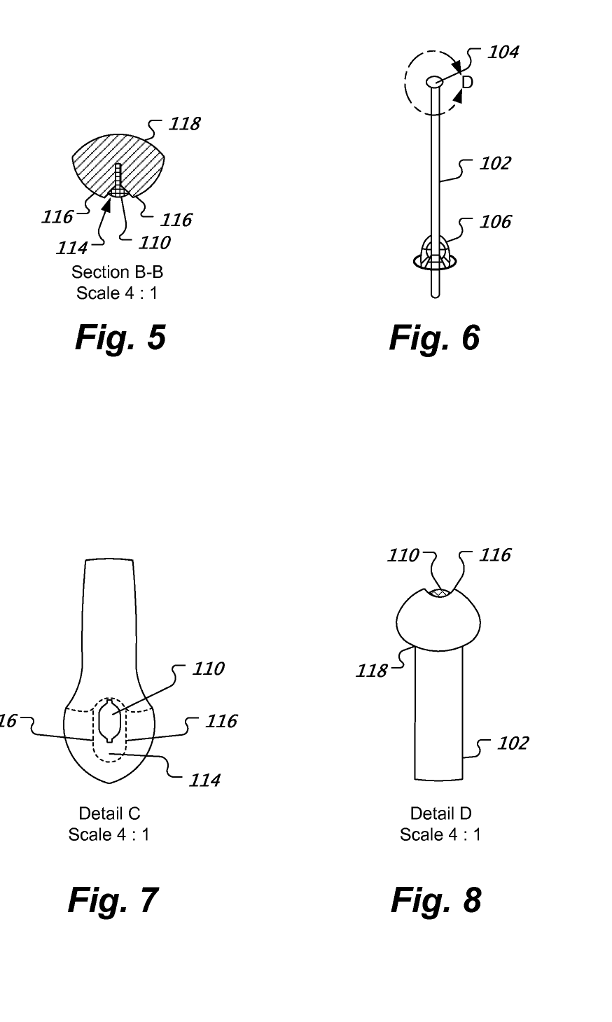

FIG. 5 is cross-sectional view of the blunt tip 104 viewed from its front, along line B-B shown in FIG. 2. FIG. 5 shows that the blunt tip may be rounded and that one side of blunt tip 104 may have a recessed area 114 that may contain all, or at least a substantial portion, of the surgical cutting element 110. It is possible that the surgical cutting element would not be contained entirely within the recessed area 114; for example, the surgical cutting element 110 may extend outwardly so that it extends vertically outside of the recessed area, or part of the surgical cutting element 110 may be positioned along the surface of the blunt tip 104 extending laterally beyond the boundary of the recessed area 114. The side of the blunt tip 104 with the recessed area 114 and surgical cutting element 110 may be referred to as the cutting surface 116. In this embodiment, the surgical cutting element 110 is approximately flush with the cutting surface 116. The surgical cutting element 110 may be provided in a recess of the blunt tip 104 to prevent the surgical cutting element from snagging on, for example, breast tissue or other structures within the patient's body. In addition, as can be seen in FIG. 5, providing the recessed area 114 and the surgical cutting element 110 on the cutting surface 116 directs the surgical cutting element 110 away from the non-cutting surface 118 and thereby isolates the surgical cutting surface 116 from the non-cutting surface 118. This configuration allows for the surgeon to make incisions only on one side of the blunt tip 104. If the surgical device 100 is being used for breast surgery, for example, configuring the surgical device 100 such that incisions are made on one side of the blunt tip 104 allows the surgeon to cut away from the implant to protect the implant and to avoid contact with tissues on which cutting is not desired. The surgeon may accomplish this goal by directing the cutting surface 116 to the desired tissue and the non-cutting surface 118 to the implant and/or tissues that should not be cut. Moreover, positioning the cutting element 110 and cutting surface 116 on one side of the blunt tip 104 focuses cutting energy at the desired point of contact. Otherwise, the energy may not be focused properly and may therefore be ineffective.

FIG. 6 is a front view of the surgical device 100. FIG. 7 is a top view of the blunt tip 104 taken along line C shown in FIG. 2, while FIG. 8 is a front view of the blunt tip 104 taken along line D shown in FIG. 6. FIGS. 7 and 8 show that the surgical cutting element 110 may be disposed in the recessed area 114. FIGS. 6 and 7 also illustrate, with the surgical cutting element 110 disposed within the recessed area 114 on the surgical cutting surface 116 and directed away from the non-cutting surface 118.

The invention will now be described with reference to an exemplary surgical procedure, called percutaneous minimally invasive capsulotomy (or “Pmic”). Currently, there have been more than 3 million breast augmentation procedures (six million implants) performed in the United States since the advent of the silicone breast implant in the 1960s. The most common complication continues to be capsular contracture, which is a tightening of the normal scar tissue interface that surrounds the implant, resulting in a firm, hard breast. This complication occurs in 14-17% of patients followed for four years after implantation (data from Allergan) and continues to occur at a similar rate for the lifespan of the implant. The current treatments are non-surgical and surgical. The current use of a drug called Singulair helps a small percentage of capsular contractures, but the majority requires surgical intervention. This is performed by either cutting the capsule (capsulotomy) or removing the capsule (capsulectomy). Both procedures usually require making a 3-4 cm incision in the breast, removing the implant, and either cutting or removing the capsule, followed by reinserting the implant, and closure of the deep tissues and the incision. It is often performed under general anesthesia and requires a few days of post-operative recovery.

Pmic is a exemplary procedure that may use an embodiment of the inventive surgical device 100 of the present invention. The surgical device 100 is used to accomplish capsulotomy through a very small (less than one cm) incision and requires minimal disruption to the breast tissues. This procedure can be performed under local anesthesia, the implant remains intact, and there is almost no post-operative recovery.

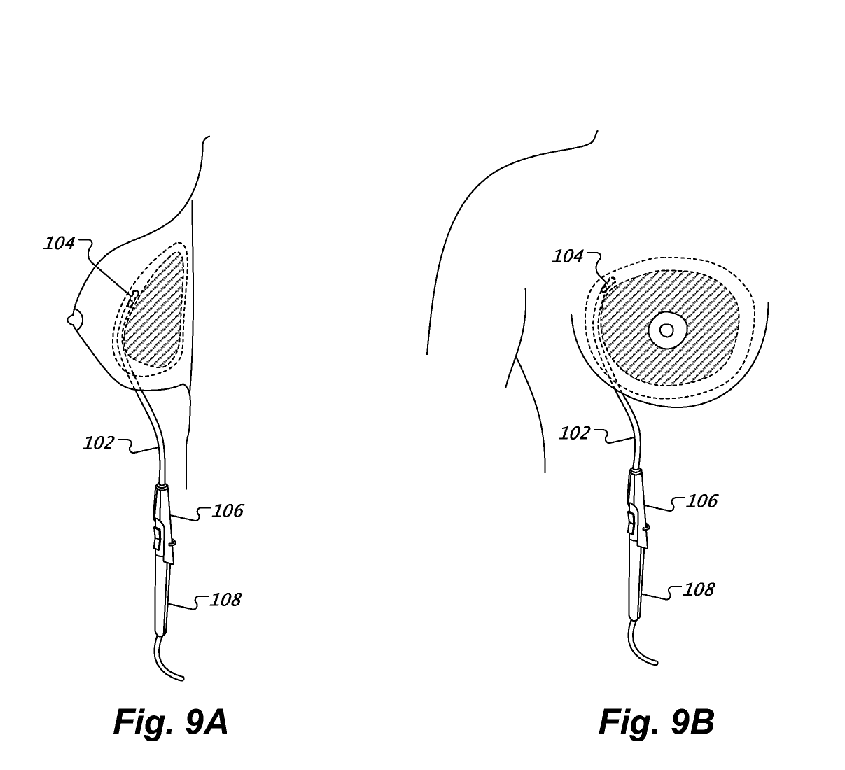

In performing Pmic, a surgeon my use an embodiment of the surgical device 100 that employs a radio-frequency cautery element (surgical cutting element 110) with a surrounding blunt tip 104 that can enter into the cavity surrounding the breast implant through a small stab incision. The blunt tip 104 allows the instrument to be passed between the implant and the surrounding scar tissue (capsule) without snagging or injuring the implant. The cautery element 110 can then be directed to cut the capsule at any point that is considered necessary or appropriate. By cutting the capsule there is an instant release of the pressure exerted on the implant and a softening of the breast. After removal of the surgical device 100 the incision is closed with a single suture. The procedure can be further understood by reference to FIGS. 9A and 9B. FIG. 9A is a side view showing an exemplary embodiment of the surgical device 100 inserted into a patient's breast, where the blunt tip 104 is applied to the scar tissue (capsule). FIG. 9B is a front view of a patient's breast showing an exemplary embodiment of the surgical device 100 as it used to cut the scare tissue (capsule). Note that the shaft 102 is gently curved to facilitate movement of the blunt tip 104 around the breast implant and scar tissue.

The typical Pmic procedure would start by numbing the access site with local anesthetic, and then a series of rib blocks would be performed—injecting each intercostal nerve with local anesthesia to numb the entire anterior chest and breast. The patient would then be sterilely prepped and draped as any normal surgical procedure. A small stab incision is then made on the under surface of the breast in the inframammary crease, and the blunt tip 104 of surgical device 100 is utilized to pierce the periprosthetic capsular tissue without injuring the implant. The surgical device 100 is connected with a standard cautery device (an embodiment of the energy delivery mechanism 108), then inserted and navigated around the breast implant with the cutting surface 114 oriented away from the implant and in contact with the capsule. The blunt tip 104 is pressed against the capsule as the cautery button on the cautery device 108 is depressed, and the cutting surface 114 of the blunt tip 104 is pulled along the capsule with the cautery button depressed cutting the capsular tissue. By applying the cutting surface 114 of the blunt tip 104, and hence the cautery element 110, to the capsular tissue, the surgeon is able to make cuts in the capsular tissue. The surgeon may repeat the process multiple times with multiple passes to ensure that the tissue is adequately cut. The surgical device 100 is then removed and a breast dissector is placed to stretch the areas that have been cut. After the removal of the breast dissector, the breast is manually compressed to assess the adequacy of the release and further stretch and release the areas of the capsule which have been cut. At the completion of the procedure there is a mandatory wait time of 5 minutes to assess for bleeding. A red rubber type catheter is then placed into the breast cavity, and the cavity is irrigated with approximately 100 cc of sterile solution followed by the placement of a blunt tipped Yankhauer suction again to assess for any bleeding. When convinced that there is no bleeding, the access incision is closed with a single suture. The incision is dressed with a Band-Aid, and the patient's breast is wrapped in a standard compressive dressing. Total operative time is about 12 minutes.

Spine surgery is another exemplary procedure that may use an embodiment of the inventive surgical device 100 of the present invention. The surgical device 100 can be used to cut tissue around a nerve or the nerve root. The surgical device 100 may be used to protect the nerve or nerve root by exposing the nerve or nerve root only to the non-cutting surface 118 of the blunt tip 104, while applying the cutting surface 114 to the scar tissue while incising that tissue. Yet another exemplary procedure in which the surgical device 100 may be used is orthopedic surgery. For example, the surgical device 100 may be used for heat shrinking of lining or capsule of a joint, for example, a knee joint.

A number of embodiments of the invention have been described. Nevertheless, it will be understood that various modifications may be made without departing from the spirit and scope of the invention. For example, when used in spinal surgery the blunt tip 104 may be narrowed so that it will fit into a smaller space, or the blunt tip 104 may be enlarged for shrinkage of a joint. As another example, the cutting element 110 may be more rounded to diffuse heat instead of cutting. Yet another example of a modification would be to include a light element on or near the blunt tip 104 or at the end of the shaft 102 so that the surgery can be visualized outside the body. Similarly, a camera or scope may be included on or near the blunt tip 104 for endoscopic visualization. In addition, the surgical device 100 may include suction at or near the blunt tip 104 or on the shaft 102 to suck out fluids during surgery, and for better adhesion to target tissues that are being cut or heated. Accordingly, other embodiments are within the scope of the following claims.

What is claimed is:

1. An apparatus for performing minimally invasive surgery, comprising:
a) a shaft having a first end and a second end, the shaft being generally stiff with a degree of flexure;
b) a blunt tip at the second end of the shaft, the blunt tip being larger in diameter than the shaft and having a cutting surface and a non-cutting surface, the blunt tip having a recessed area at least substantially along the cutting surface;
c) a flange located at the second end of the shaft, the flange configured to receive a device that delivers an energy;
d) an energy conductor disposed along the length of the shaft from the second end of the shaft to at least proximate the recessed area, the energy conductor configured to conduct the energy; and
e) a surgical cutting element located at least substantially within the recessed area of the blunt tip and coupled to the energy conductor, the surgical cutting element configured to make incisions in response to receiving the energy from the energy conductor.

2. The apparatus recited in claim 1 wherein the shaft is curved along at least a substantial part of the length of the shaft from the first end to the second end.

3. The apparatus recited in claim 1 wherein the energy is radio-frequency and the surgical cutting element is a cautery element.

4. The apparatus recited in claim 3 wherein the cautery element is disposed at least substantially within the recessed area of the cutting surface of the blunt tip and is configured to be approximately flush with the cutting surface to allow the cautery element to engage tissue to make incisions.

5. The apparatus recited in claim 4 wherein the cautery element is directed away from the non-cutting surface of the blunt tip.

6. The apparatus recited in claim 5 wherein the flange is configured to receive a standard cautery device.

7. The apparatus recited in claim 1 wherein the energy is laser light and the surgical cutting element emits a laser beam.

8. The apparatus recited in claim 7 wherein the flange is configured to receive a standard laser surgery device.

9. An apparatus for performing minimally invasive surgery, comprising:
a) a shaft having a first end and a second end, the shaft being generally stiff with a degree of flexure;
b) a blunt tip means, located at the second end of the shaft and having a cutting surface and a non-cutting surface, for inserting into a patient's body through an incision;
c) a flange means, located at the second end of the shaft, for receiving a device that delivers an energy;
d) a conductor means, disposed along the length of the shaft from the second end of the shaft to at least proximate the blunt tip means, for conducting the energy; and
e) a cutting means, located at least partially on the cutting surface of the blunt tip means and responsive to the energy from the conductor means, for making incisions from the cutting surface of the blunt tip means and away from the non-cutting surface of the blunt tip means.

10. The apparatus recited in claim 9 wherein the energy is radio-frequency and the cutting means is a cautery element.

11. The apparatus recited in claim 10 wherein the cautery element is disposed at least substantially within a recessed area located at the cutting surface of the blunt tip means and is configured to be approximately flush with the cutting surface to allow the cautery element to contact tissue and thereby make incisions.

12. The apparatus recited in claim 11 wherein the cautery element is directed away from the non-cutting surface of the blunt tip means.

13. The apparatus recited in claim 12 wherein the flange means is configured to receive a standard cautery device.

14. The apparatus recited in claim 9 wherein the energy is laser light and the cutting means emits a laser beam.

15. The apparatus recited in claim 14 wherein the flange means is configured to receive a standard laser surgery device.

16. A minimally invasive method for performing capsulotomy surgery, comprising:
a) providing a surgical device having a shaft, a blunt tip, and a flange, and wherein:
i) the shaft has a first end and a second end, the shaft being generally stiff with a degree of flexure,
ii) the blunt tip is located at the second end of the shaft and is larger in diameter than the shaft and has a cutting side and a non-cutting side, the blunt tip having a recess located at least substantially on the cutting side,
iii) a surgical cutting element is located at least substantially within the recess of the blunt tip and is directed away from the non-cutting side,
iv) an energy conductor is disposed along the length of the shaft from the second end of the shaft to the surgical cutting element and is configured to deliver an energy for use by the surgical cutting element, and
v) the flange is located at the second end of the shaft and is configured to receive a device that delivers the energy to the energy conductor;
b) inserting the blunt tip of the surgical device into an incision on or near a breast of a patient and into a cavity surrounding a breast implant within the breast;
c) passing the blunt tip between the implant and surrounding scar tissue and directing the cutting side and the surgical cutting element toward the scar tissue and away from the implant; and
d) cutting the scar tissue with the surgical cutting element.

17. The minimally invasive method for performing capsulotomy surgery of claim 16, wherein the shaft is curved, the method further comprising using the curve of the shaft to facilitate navigation of the blunt tip around the breast implant.

18. The minimally invasive method for performing capsulotomy surgery of claim 16, further comprising:
a) conducting a radio-frequency energy through the energy conductor to the surgical cutting element; and
b) cauterizing the scar tissue with the surgical cutting element.

19. A minimally invasive method for performing spinal surgery, comprising:
a) providing a surgical device having a shaft, a blunt tip, and a flange, and wherein:
i) the shaft has a first end and a second end, the shaft being generally stiff with a degree of flexure,
ii) the blunt tip is located at the second end of the shaft and is larger in diameter than the shaft and has a cutting side and a non-cutting side, the blunt tip having a recess located at least substantially on the cutting side,
iii) a surgical cutting element is located at least substantially within the recess of the blunt tip and is directed away from the non-cutting side,
iv) an energy conductor is disposed along the length of the shaft from the second end of the shaft to the surgical cutting element and is configured to deliver an energy for use by the surgical cutting element, and
v) the flange is located at the second end of the shaft and is configured to receive a device that delivers the energy to the energy conductor;
b) inserting the blunt tip of the surgical device into an incision on or near a patient's spine;
c) directing the blunt tip toward a scar tissue disposed at or near a nerve or nerve root;
d) directing the cutting surface of the blunt tip toward the scar tissue and the non-cutting surface of the blunt tip toward the nerve or nerve root; and
e) cutting the scar tissue with the cutting element of the surgical device.

20. A minimally invasive method for performing joint surgery, comprising:
a) providing a surgical device having a shaft, a blunt tip, and a flange, and wherein:
i) the shaft has a first end and a second end, the shaft being generally stiff with a degree of flexure,
ii) the blunt tip is located at the second end of the shaft and is larger in diameter than the shaft and has a heating side and a non-heating side, the blunt tip having a recess located at least substantially on the heating side,
iii) a surgical heating element is located at least substantially within the recess of the blunt tip and is directed away from the non-heating side,
iv) an energy conductor is disposed along the length of the shaft from the second end of the shaft to the surgical heating element and is configured to deliver an energy for use by the surgical heating element, and
v) the flange is located at the second end of the shaft and is configured to receive a device that delivers the energy to the energy conductor;
b) inserting the blunt tip of the surgical device into an incision on or near a patient's joint;
c) directing the blunt tip toward a lining or capsule of the joint; d) directing the heating surface of the blunt tip toward the lining or capsule and the non-heating surface of the blunt tip away from the lining or capsule; and
e) heating the lining or capsule with the surgical heating element of the surgical device.

21. An apparatus for performing minimally invasive surgery, comprising:
a) a shaft having a first end and a second end, the shaft being generally stiff with a degree of flexure;
b) a blunt tip at the second end of the shaft, the blunt tip being larger in diameter than the shaft and having an active surface and a non-active surface, the blunt tip having a recessed area at least substantially along the active surface;
c) a flange located at the second end of the shaft, the flange configured to receive a device that delivers an energy;
d) an energy conductor disposed along the length of the shaft from the second end of the shaft to at least proximate the recessed area, the energy conductor configured to conduct the energy; and
e) an active surgical element located at least substantially within the recessed area of the rounded blunt tip and coupled to the energy conductor, the active surgical element configured to surgically modify tissue in response to receiving the energy from the energy conductor.

22. The apparatus recited in claim 21 wherein the active surgical element is a surgical cutting element.

23. The apparatus recited in claim 22 wherein the energy is radio-frequency and the surgical cutting element is a cautery element.

24. The apparatus recited in claim 23 wherein the cautery element is disposed at least substantially within the recessed area of the active surface of the blunt tip and is configured to be approximately flush with the active surface to allow the cautery element to engage tissue to make incisions.

25. The apparatus recited in claim 24 wherein the cautery element is directed away from the non-active surface of the blunt tip.

26. The apparatus recited in claim 25 wherein the shaft is curved at least substantially along its length from the first end to the second end.

27. The apparatus recited in claim 22 wherein the energy is laser light and the surgical cutting element emits a laser beam.

28. The apparatus recited in claim 21 wherein the active surgical element is a surgical heating element.
